(19) United States
(12) Patent Application Publication
(10) Pub. No.: US 2022/0047166 A1
(43) Pub. Date: Feb. 17, 2022

(54) BIOLOGICAL INFORMATION DETECTOR

(71) Applicant: SOUKEN CO., LTD., Hiroshima (JP)

(72) Inventor: Hiroto MASUDA, Hiroshima (JP)

(21) Appl. No.: 17/059,515

(22) PCT Filed: Jul. 22, 2020
(86) PCT No.: PCT/JP2020/028545

§ 371 Date: Nov. 30, 2020

(30) Foreign Application Priority Data
Feb. 25, 2020 (JP) 2020-029225

(51) Int. Cl. A61B 5/00; A61B 5/28; A61B 5/145; A61B 5/026; A61B 5/08; A61B 5/021

(52) U.S. Cl. CPC A61B 5/0088; A61B 5/682; A61B 5/28; A61B 5/02141; A61B 5/026; A61B 5/08; A61B 5/14542

(57) ABSTRACT

A sensor holder 20 holding a biological information detection sensor 10 for detecting biological information in an oral cavity includes an attachment portion 21 to be attached to the tongue. The attachment portion 21 is continuous to surround the front surface, lateral surfaces, and back surface of the tongue of a subject.

### TECHNICAL FIELD

The present invention relates to a biological information detector that detects biological information such as vital signs, for example.

### BACKGROUND ART

A monitoring system has been known that obtains vital signs of a patient subjected to a certain treatment, and obtains an index of an effect obtained by the treatment performed on the patient based on the obtained vital signs (e.g., see Patent Document 1). In the system of Patent Document 1, a vital sensor includes a pulse oximeter, a respiratory sensor that measures a respiratory rate, a blood pressure sensor that measures a blood pressure, a thermometer that measures a body temperature, a pulse wave sensor that measures pulse waves, a heart rate sensor that measures a heart rate, and other sensors as usable sensors.

### CITATION LIST

### Patent Document

PATENT DOCUMENT 1: Japanese Unexamined Patent Publication No. 2019-122476

### SUMMARY OF THE INVENTION

### Technical Problem

Since the oral cavity of a living body has a mucous membrane that is thinner than the skin, and blood vessels are dense in the tongue, a highly accurate detection may be performed by inserting a sensor in the oral cavity and detecting biological information such as vital signs. However, for the measurement with the sensor to detect the biological information, the arrangement of the sensor at a measurement position in advance is problematic. Specifically, it is conceivable that the sensor is held by a finger with the sensor being inserted into the oral cavity, but in this case, a mouth must be opened for a long period of time, so that this is a difficult way to fix the sensor. Therefore, the sensor might be displaced during the measurement, thereby deteriorating measurement accuracy.

Further, the arterial blood oxygen saturation and pulse rate are measured with a pulse oximeter in various medical institutions, nursing care institutions, and the like, and measurement sites with the pulse oximeter are, for example, fingertips of limbs and an ear. The measurement may be difficult due to an influence of manicure on nails of the limbs, which are the measurement sites. Furthermore, there is a problem that an error is likely to occur in the measurement with the pulse oximeter in the case where the blood flow is obstructed, or peripheral circulation failure occurs due to pressing of an arm or a finger, and the case where ambient light is too strong.

In view of the forgoing, it is an object of the present invention to enable detection of the biological information in the oral cavity with high accuracy.

### Solution to the Problem

In order to achieve the above object, a first disclosure is directed to a biological information detector including a biological information detection sensor configured to be inserted into an oral cavity and detect biological information in the oral cavity, and a sensor holder holding the biological information detection sensor. The sensor holder includes an attachment portion continuous to surround a front surface, lateral surfaces, and a back surface of a tongue of a subject, and is used to attach the tongue.

With this configuration where the attachment portion attached to the tongue is continuous to surround the front surface, lateral surfaces, and back surfaces of the tongue, the attachment portion is less likely to move upward, downward, or laterally from the tongue. Further, this attachment portion holds the biological information detection sensor. Thus, the biological information detection sensor attached to the attachment portion so as to be arranged at a measurement position set in advance is less likely to move from the measurement site during the measurement.

In a second disclosure, the sensor holder further includes an extending portion continuous from the attachment portion toward an area between upper and lower teeth of the subject.

With this configuration, the subject may bite and hold the extending portion of the sensor holder with his/her upper and lower teeth, which allows the movement of the attachment portion in the oral cavity to be reduced.

In a third disclosure, the attachment portion has an annular shape continuous to surround the front surface, left lateral surface, back surface, and right lateral surface of the tongue of the subject.

With this configuration where the attachment portion is continuous to surround the tongue, the attachment portion is less likely to move upward, downward, leftward, or rightward from the tongue.

In a fourth disclosure, an inflation member that presses the tongue is arranged in an inner peripheral surface of the attachment portion, and the biological information detection sensor is a blood pressure sensor including the inflation member.

With this configuration where the attachment portion has an annular shape, when the inflation member is inflated with the attachment portion attached to the tongue, an inflating force of the inflation member is less likely to escape, thereby allowing the inflation member to reliably press the tongue. This pressing partially stops a blood flow of the tongue, and when the inflation member is then gradually deflated until the blood flows again, a small heartbeat (pulse phenomenon) may be detected. This pulsation becomes larger as tightening by the inflation member becomes looser, reaches the largest amplitude, and then becomes smaller again. The blood pressure may be calculated by analysis of amplitude waveform information of this pulsation with a predetermined algorithm. Specifically, the blood pressure may be measured by an oscillometric system using the tongue.

In a fifth disclosure, the biological information detection sensor includes a light emitter that irradiates the tongue with light, and a light receiver that receives the light applied to the tongue from the light emitter, the light emitter and the light receiver being arranged to come into contact with the back surface of the tongue.

With this configuration, the light emitter and the light receiver are held so as not to move from the back surface of the tongue. Then, the light receiver receives the light with the tongue irradiated with the light from the light emitter. The arterial blood oxygen saturation, pulse wave, and other vital signs may be detected based on a change in light received by the light receiver.

In a sixth embodiment, the biological information detection sensor includes a light emitter that irradiates a gum with light, and a light receiver that receives the light applied to the gum from the light emitter, the light emitter and the light receiver being arranged so as to face the gum.

With this configuration, the light emitter and the light receiver are held so as not to move from predetermined positions. Then, the light receiver receives the light with the gum irradiated with the light from the light emitter. For example, a state of the gingivae may be detected based on a change in the light received by the light receiver.

In a seventh aspect, the biological information detection sensor is an electrocardiographic measurement sensor including an intraoral electrode arranged to come into contact with the tongue and an extraoral electrode arranged outside the oral cavity to come into contact with a hand of the subject.

With this configuration, an electric circuit including the intraoral electrode and the extraoral electrode is formed. This electric circuit allows electricity in the art to be detected, thereby obtaining an electrocardiogram.

In an eighth disclosure, the biological information detection sensor is an expired gas sensor or a respiratory sound sensor.

With this configuration, the expired gas sensor may be arranged in the oral cavity. This arrangement allows components contained in expiration to be reliably detected. Furthermore, since the respiratory sound sensor may be arranged in the oral cavity, the respiratory sound of the subject may be reliably detected.

### Advantages of the Invention

As described, in the present disclosure, a sensor holder holding a biological information detection sensor configured to detect biological information in an oral cavity includes an attachment portion continuous to surround a front surface, lateral surfaces, and a back surface of the tongue of the subject. This configuration allows the biological information detection sensor to be arranged so as not to move from a measuring position during measurement, and allows the biological information in the oral cavity to be detected with high accuracy.

### BRIEF DESCRIPTION OF THE DRAWINGS

FIG. 1 is a perspective view of a biological information detector according to a first embodiment of the present invention.

FIG. 2 is a side view of the biological information detector according to the first embodiment.

FIG. 3 is an explanatory diagram illustrating the biological information detector according to the first embodiment in use in an oral cavity and its vicinity of a subject.

FIG. 4 is a block diagram of a detection device including the biological information detector according to the first embodiment.

FIG. 5 is a perspective view of a biological information detector according to a second embodiment of the present invention.

FIG. 6 is an explanatory diagram illustrating the biological information detector according to the second embodiment in use in an oral cavity and its vicinity of a subject.

FIG. 7 is a block diagram of a detection device including the biological information detector according to the second embodiment.

FIG. 8 is a perspective view of a biological information detector according to a third embodiment of the present invention.

FIG. 9 is a side view of the biological information detector according to the third embodiment.

FIG. 10 is a block diagram of a detection device including the biological information detector according to the third embodiment.

FIG. 11 is a perspective view of a biological information detector according to a fourth embodiment of the present invention.

FIG. 12 is a cross-sectional view of the biological information detector according to the fourth embodiment.

FIG. 13 is a block diagram of a detection device including the biological information detector according to the fourth embodiment.

FIG. 14 is a perspective view of a biological information detector according to a fifth embodiment of the present invention.

FIG. 15 is a cross-sectional view of the biological information detector according to the fifth embodiment.

FIG. 16 is an explanatory diagram illustrating the biological information detector according to the fifth embodiment in use in an oral cavity and its vicinity of a subject.

FIG. 17 is a block diagram of a detection device including the biological information detector according to the fifth embodiment.

### DESCRIPTION OF EMBODIMENTS

Embodiments of the present invention will be described in detail with reference to the drawings. The following description of preferred embodiments is a mere example in nature, and is not intended to limit the scope, applications or use of the present invention.

### First Embodiment

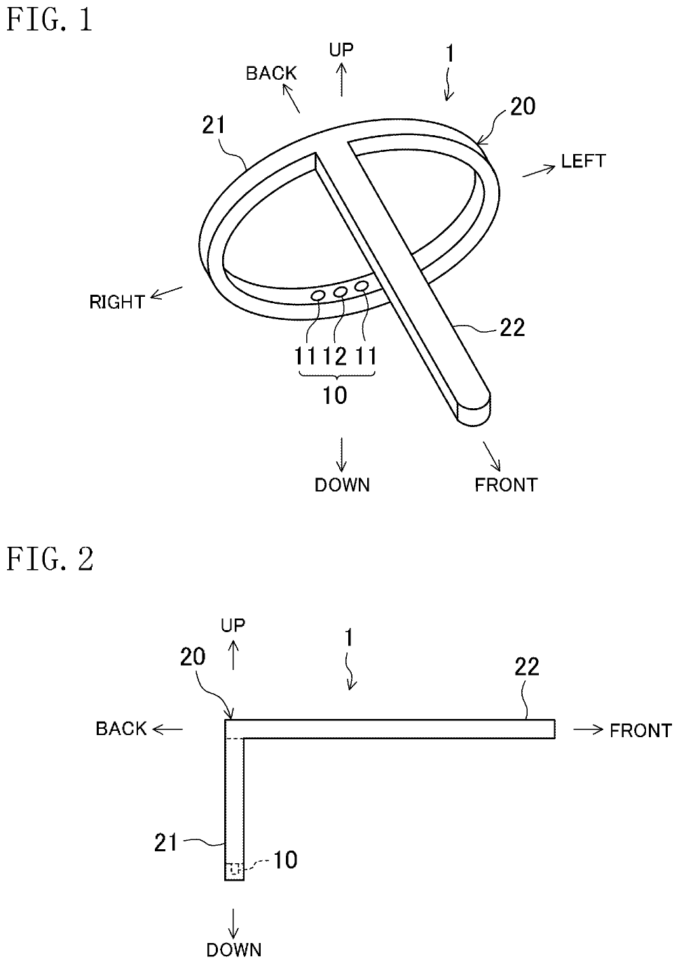

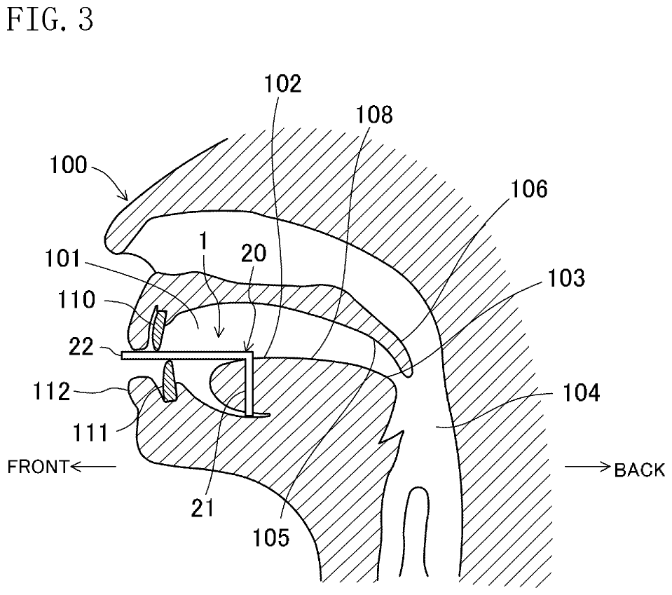

FIG. 1 is a perspective view of a biological information detector 1 according to a first embodiment of the present invention. FIG. 2 is a side view of the biological information detector 1. As also illustrated in FIG. 3, the biological information detector 1 includes: a biological information detection sensor 10 configured to be inserted into an oral cavity 101 of a subject 100, and detect biological information in the oral cavity 101; and a sensor holder 20 holding the biological information detection sensor 10. In the description of this embodiment, with the use of the biological information detector 1, that is, with the biological information detection sensor 10 being inserted into the oral cavity 101, the front portion in an insertion direction is referred to as the front, and the back portion in the insertion direction is referred to as the back. Thus, a front portion of the biological information detector 1 is a portion located to correspond to front teeth 110 and 111 of the subject 100, and a back portion is a portion located to correspond to a tongue base 103. Further, with the biological information detection sensor 10 being inserted into the oral cavity 101, a right side of the subject 100 is merely referred to as the right, and a left side of the subject 100 is merely referred to as the left.

The biological information includes information indicating a physical condition and vital signs. Examples of the vital signs include a measurement value of arterial blood oxygen saturation, body temperature, heart rate, pulse, blood pressure, and blood oxygen level, and the vital signs are signals indicating that a person is alive, and signals indicating whether a person is in a normal state. Furthermore, the subject 100 may be a healthy person, an inpatient, a home-care patient, or a person who receives nursing care. Therefore, a place where the biological information detector 1 is used may be a home, a medical institution, and a nursing care institution.

FIG. 3 illustrates the oral cavity 101 and the vicinity thereof of the subject 100. About one-third on the back portion of the tongue 102 is the tongue base 103, and about two-thirds of the front of the tongue base 103 is a tongue movable portion (tongue body) 108. A muscle for changing the position of the tongue 102 is referred to as an extrinsic tongue muscle. The extrinsic tongue muscle includes: a styloglossus muscle pulling the tongue 102 toward the back; a hyoglossus muscle pulling the tongue 102 downward; a palatoglossus muscle provided to an external rim of the tongue and lifting the tongue dorsum; and a genioglossus muscle projecting the tongue 102 forward. Further, there is an airway 104 behind a soft palate 105 and a uvula of soft palate 106. FIG. 3 further illustrates the upper front tooth 110, the lower front tooth 111, and a lip 112.

### (Configuration of Sensor Holder 20)

As illustrated in FIGS. 1 and 2, the sensor holder 20 includes an attachment portion 21 and an extending portion 22. The attachment portion 21 and the extending portion 22 may be formed from, for example, a resin material. The attachment portion 21 and the extending portion 22 may be integral with each other, or the attachment portion 21 and the extending portion 22 formed from different members may be integral with each other. The resin material that forms the attachment portion 21 and the extending portion 22 may be an elastic resin material, a soft resin material, or a hard resin material. The attachment portion 21 and the extending portion 22 may be formed from different resin materials.

The attachment portion 21 has an annular shape. Specifically, the attachment portion 21 is continuous to surround a front surface (upper surface), a left lateral surface, a back surface (lower surface), and a right lateral surface of the tongue 102 of the subject 100. The attachment portion 21 may be formed into an elliptical or oval shape continuing in a right-to-left direction so as to correspond to a cross-sectional shape of the tongue 102 in the right-to-left direction. The attachment portion 21 may be circular, but in this case, the attachment portion 21 is formed from preferably a material which is easily deformable by insertion of the tongue 102.

An inner perimeter of the attachment portion 21 is set to be substantially equal to a perimeter of an intermediate portion in a front-to-back direction of a tongue movable portion 108 of the tongue 102. This configuration allows the tongue movable portion 108 inserted into the attachment portion 21 from the tip thereof to be further inserted into the attachment portion 21 up to the intermediate portion thereof when the attachment portion 21 is attached to the tongue 102, thereby allowing the attachment portion 21 to be attached to the tongue movable portion 108 easily. Among a plurality of types of biological information detectors 1 having different perimeters of the attachment portion 21, a biological information detector 1 having a perimeter which fits the tongue movable portion 108 of the subject 100 may be selected.

The attachment portion 21 may also be formed from a resin band or a stripe member. In this case, the perimeter of the attachment portion 21 may be adjusted according to the perimeter of the tongue movable portion 108 of the subject 100. For the attachment portion 21 formed from an elastic material such as rubber or elastomer, for example, when the tongue movable portion 108 of the subject 100 is inserted into the attachment portion 21, the attachment portion 21 extends to fit the perimeter of the tongue movable portion 108, so that the inner peripheral surface of the attachment portion 21 comes into close contact with the tongue movable portion 108.

The width of the attachment portion 21 may be set within a range of 2 mm or more to 20 mm or less, for example. The width of the attachment portion 21 may be set according to the type, number, and other parameters of the biological information detection sensors 10 to be described later. The attachment portion 21 may have an annular shape continuous in a circumferential direction, or a part of the annular shape in the circumferential direction may be discontinuous.

The extending portion 22 has a rod shape or a plate shape extending forward from an upper portion and a central portion of the attachment portion 21 in the right-to-left direction. The extending portion 22 is continuous from the attachment portion 21 toward an area between the upper front tooth (upper tooth) 110 and the lower front tooth (lower tooth) 111 of the subject 100. A front portion of the extending portion 22 is a portion that is fixable by being bitten with the upper front tooth 110 and the lower front tooth 111 of the subject 100 from above and below. A front end (distal end) of the extending portion 22 may be located inside or outside the oral cavity 101 of the subject 100. The extending portion 22 formed from a hard resin material allows deformation caused when bitten with the front teeth 110 and 111 to be reduced. A thickness (dimension in the up-and-down direction) of the extending portion 22 may be set within a range of 1 mm or more to 5 mm or less, for example. A dimension in the right-to-left direction of the extending portion 22 may be set within a range of 1 mm or more to 30 mm or less, for example. The extending portion 22 may be provided as required and may be omitted. A plurality of extending portions 22 may be provided.

### (Configuration of Biological Information Detection Sensor 10)

The biological information detection sensor 10 includes two light emitting elements (light emitters) 11 that irradiate the tongue movable portion 108 with light, and a light receiving element (light receiver) 12 that receives the light applied to the tongue movable portion 108 from the light emitting elements 11, the elements being arranged so as to come into contact with the back surface of the tongue movable portion 108 of the tongue 102. The light emitting elements 11 may be, for example, light emitting elements typically used for blood flow measurement and other measurements, such as light emitting diodes that apply infrared light. The light receiving element 12 may be a light receiving element such as a photodiode also typically used for blood flow measurement and other measurements. The light to be applied from the tongue movable portion 108 may be, for example, but is not limited to, near-infrared light, and may be any light capable of detecting the arterial blood oxygen saturation, a pulse rate, and parameters.

The light emitting elements 11 are attached to the attachment portion 21 so as to apply light upward at a lower side of the inner peripheral surface of the attachment portion 21. The light emitting elements 11 provided on the lower side of the inner peripheral surface of the attachment portion 21 allow the light applied from the light emitting elements 11 to reliably reach the back surface of the tongue movable portion 108. In this embodiment, the light emitting elements 11 are arranged such that their front surfaces come into contact with the back surface of the tongue movable portion 108. Many arteries run on the back surface of the tongue movable portion 108, and the arteries and tissues in the vicinity thereof may be illuminated by the light emitting elements 11. Only one light emitting element 11 may be provided, or three or more of the light emitting elements 11 may be provided. For a plurality of light emitting elements 11 provided, they are arranged at intervals in the circumferential direction or the width direction of the attachment portion 21 in one preferred embodiment.

The light receiving element 12 is also attached to the attachment portion 21 so as to be arranged on the lower side of the inner peripheral surface of the attachment portion 21 with its light receiving surface facing upward. A front surface of the light receiving element 12 is arranged so as to come into contact with the back surface of the tongue movable portion 108. Intensity of the light received by the light receiving element 12 changes depending on an arterial blood flow state, pulsation of blood, and blood oxygen saturation.

A measuring method of the blood flow and the blood oxygen saturation using light includes a reflection light method of receiving light (reflection light), the light applied from the light emitting elements 11 and reflected by the tissue or blood, and a transmission light method of using transmission light applied from the light emitting elements 11 and transmitted through the tissue or blood; either method may be used in this embodiment.

### (Configuration of Detection Device 50)

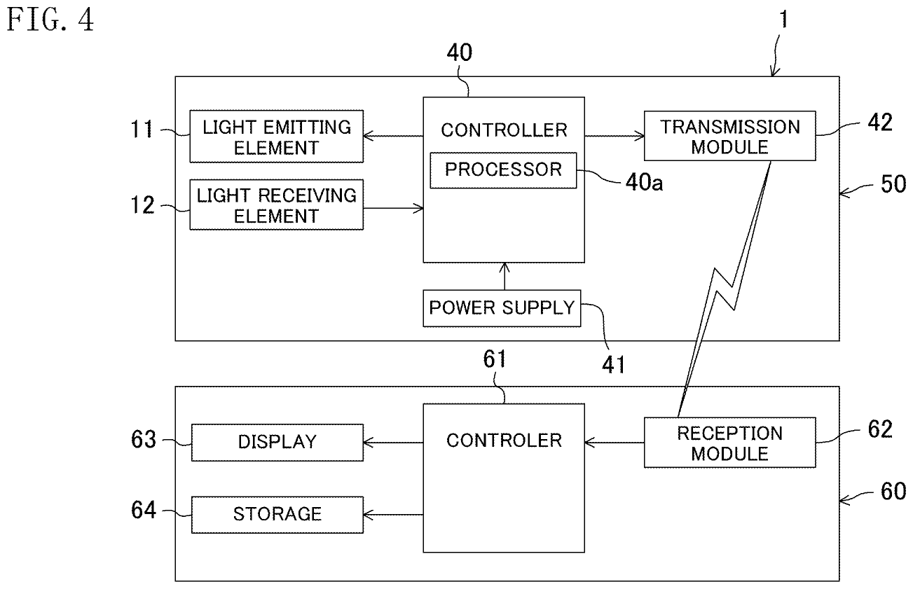

FIG. 4 is a block diagram of a detection device 50 including the biological information detector 1. The detection device 50 includes an external device 60 in addition to the biological information detector 1. The biological information detector 1 includes a controller 40, a power supply 41, and a transmission module 42 in addition to the light emitting element 11 and the light receiving element 12. The controller 40, the power supply 41, and the transmission module 42 may be embedded inside the attachment portion 21 or the extending portion 22 or may be externally arranged. For the controller 40, the power supply 41, and the transmission module 42 externally arranged, the light emitting element 11 and the light receiving element 12 may be connected to the controller 40 by a signal line. The signal line may pass through the inside of the extending portion 22 from a proximal end to the distal end.

The power supply 41 may be a small battery or a rechargeable battery, and supplies necessary power to the controller 40. The controller 40 is a portion that controls the light emitting element 11 and obtains a change in the intensity of the light received by the light receiving element 12 to convert into various vital data. For example, when the power is turned on by a switch not illustrated, the controller 40 supplies power to the light emitting element 11 to cause the light emitting element 11 to apply light. The light applied from the light emitting element 11 is received by the light receiving element 12. The intensity of the light at that time changes with time, and this change in the intensity of the light may be obtained by the light receiving element 12. The controller 40 is configured to obtain the blood flow in the tongue 102 based on the intensity of the light received by the light receiving element 12. For example, the blood flowing through a blood vessel pulsates due to the heartbeat, and when the blood vessel is irradiated with measurement light from the light emitting element 11, the intensity of the light changes corresponding to the pulsation of blood in the light receiving element 12. A processor 40a of the controller 40 may use this change to perform a predetermined calculation, thereby converting the change into the biological information such as the heartbeat, pulse, and blood oxygen level (arterial blood oxygen saturation). The change in the intensity of the light obtained by the light receiving element 12 is also a part of the biological information. Note that the method of measuring the heartbeat, pulse, and blood oxygen level through applying light is used in various devices, and there are various methods. Any configuration of them may be used in this embodiment.

The transmission module 42 is for transmitting a detection result (vital data) by the processor 40a to the external device 60. The transmission module 42 is configured to transmit the detection result to the external device 60 by a wire or wirelessly. For the wired communication, the transmission module 42 and the external device 60 may be connected to each other by a communication line. For the wireless communication, the transmission module 42 and the external device 60 may be connected so as to be able to communicate with each other by a method adhering to the existing wireless communication standards. As the method, for example, wireless LAN communication, and Bluetooth (registered trademark), which is a short-range wireless communication standard may be used. Note that the transmission module 42 may also be configured to receive a control signal from the external device 60. In this case, the external device 60 may control the controller 40.

The external device 60 includes a controller 61, a receiver module 62, a display 63, and a storage 64. Examples of devices that may be used as the external device 60 include a personal computer, a tablet terminal, and a smartphone. These terminals may be held by medical staffs, nursing staffs, the family of the subject and the like.

The receiver module 62 receives the detection result transmitted from the transmission module 42 of the biological information detector 1, and may transmit the control signal to the transmission module 42 in addition to receiving. The controller 61 makes the detection result received by the receiver module 62 into a graph, or converts the detection result into a numerical value, for example. The controller 61 may also generate a user interface screen in which the obtained graph and numerical value are incorporated. The user interface screen generated by the controller 61 is displayed on the display 63. The display 63 is, for example, a liquid crystal display panel. The detection result may also be stored in the storage 64. The storage 64 is, for example, a solid state drive (SSD), a hard disk drive, or a memory card.

The receiver module 62 is also connected to the Internet line. The detection result received by the receiver module 62 may also be uploaded to, for example, a server owned by the medical institution or the nursing care institution, for example, via the Internet line. The server may accumulate and use the detection results.

### Advantages of Embodiment

As described above, in this embodiment, the attachment portion 21 attached to the tongue 102 is continuous to surround the front surface, lateral surfaces, and back surfaces of the tongue 102. With this configuration, the attachment portion is less likely to move upward, downward, or laterally from the tongue. This attachment portion 21 holds the biological information detection sensor 10. Thus, the biological information detection sensor 10 attached to the attachment portion 21 so as to be arranged at a measurement position set in advance is less likely to move from the measurement site during the measurement. Accordingly, the biological information may be detected in the oral cavity 101 with high accuracy.

Further, the state of pulsation of blood may be obtained from the intensity of the light detected by the light receiving element 12. The blood pressure may be calculated by using a predetermined algorithm based on the state of the pulsation of blood. The blood pressure may be measured by, for example, a method loaded onto a wearable terminal.

### Second Embodiment

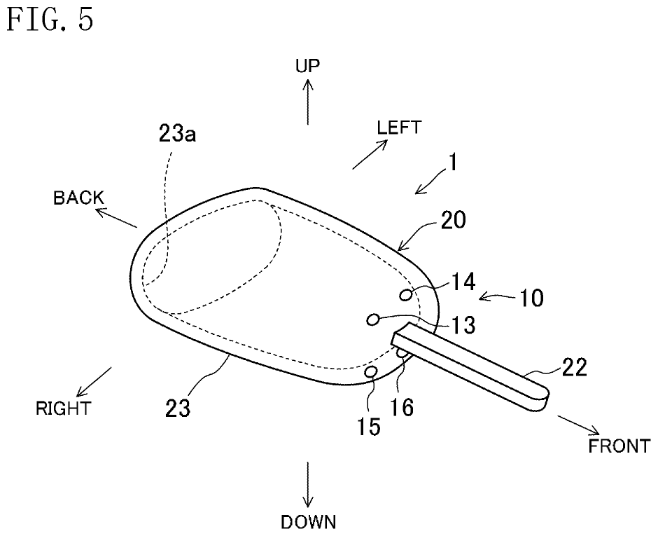

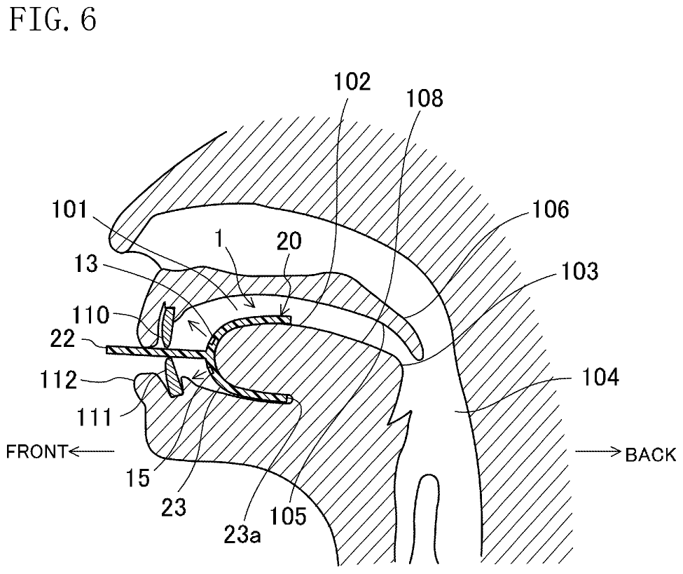

FIGS. 5 to 7 relate to a second embodiment of the present invention. The second embodiment is different from the first embodiment in that the biological information detector 1 is configured to detect the presence or absence or degree of inflammation in a deep part and gingivae in an oral cavity 101 as biological information. Hereinafter, the same parts as those of the first embodiment are denoted by the same reference numerals, and the description thereof is omitted; different parts are described in detail.

As illustrated in FIG. 5, in the second embodiment, an attachment portion 23 of a sensor holder 20 is formed into a cup shape. Specifically, the attachment portion 23 is formed such that a tongue movable portion 108 is inserted therein from the distal end up to an intermediate portion in a front-to-back direction, and an opening 23a for allowing the tongue movable portion 108 to be inserted therein is formed at a back end of the attachment portion 23. Further, the attachment portion 23 is continuous to surround the front surface, left lateral surface, back surface, right lateral surface of the tongue 102 of the subject 100, and is continuous to surround the distal end up to the intermediate portion of the tongue movable portion 108 in the front-and-back direction.

The biological information detection sensor 10 includes an upper light emitting element 13, an upper light receiving element 14, a lower light emitting element 15, and a lower light receiving element 16. The upper light emitting element 13 and the upper light receiving element 14 are provided above a central portion of the attachment portion 23 in an up-and-down direction. The upper light emitting element 13 is arranged such that a light irradiating surface thereof faces obliquely upward so as to face the upper gums and gingiva of the subject 100, and irradiates the gums and gingiva with measurement light. The upper light receiving element 14 receives the light applied to the gums and gingiva from the upper light emitting element 13, and is arranged such that a light receiving surface thereof faces obliquely upward.

The lower light emitting element 15 and the lower light receiving element 16 are provided below the central portion of the attachment portion 23 in the up-and-down direction. The lower light emitting element 15 is arranged such that a light irradiating surface thereof faces obliquely downward so as to face lower gums and gingiva of the subject 100, and irradiates the gums and gingiva with light measurement light. The lower light receiving element 16 receives the light applied to the gums and gingiva from the lower light emitting element 15, and is arranged such that a light receiving surface thereof faces obliquely downward. In the second embodiment, it is possible to set a wide light irradiation range.

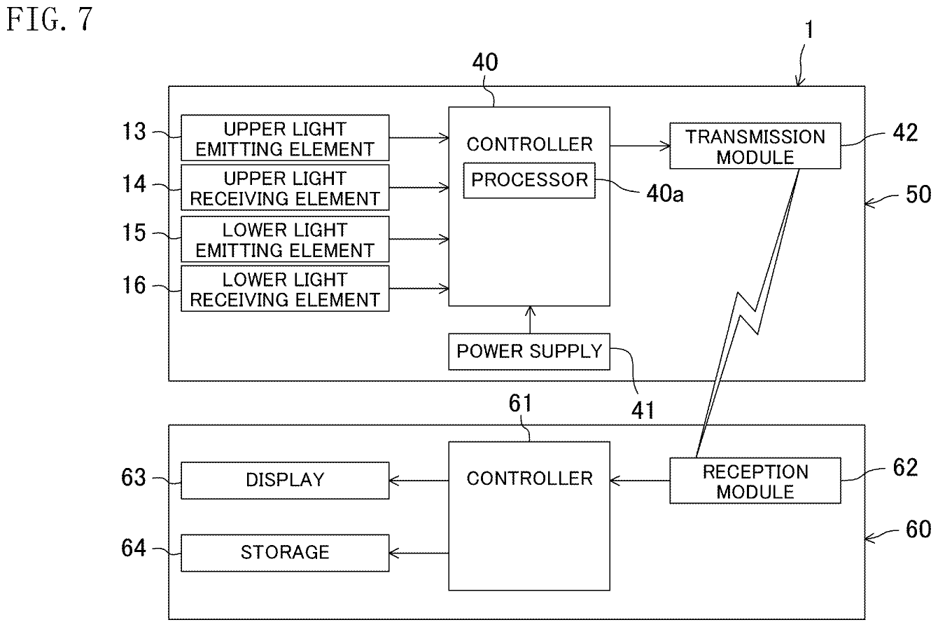

As illustrated in FIG. 7, the upper light emitting element 13, the upper light receiving element 14, the lower light emitting element 15, and the lower light receiving element 16 are connected to a controller 40. The controller 40 controls the upper light emitting element 13 and the lower light emitting element 15. The controller 40 obtains intensity of light received by the upper light receiving element 14 and the lower light receiving element 16 and converts the intensity into various vital data, and is configured to obtain the presence or absence or degree of inflammation of the gums and gingivae based on the intensity of the light in the second embodiment. For example, the degree of reflection light received when the inflamed gums and gingiva are irradiated with light, and the degree of reflection light received when the gums and inflammation without inflammation are irradiated with light are obtained in advance by experiments. Then, a correlation between the presence or absence of inflammation and the intensity of the received light is calculated. Based on the correlation and the intensity of the light received by the upper light receiving element 14 and the lower light receiving element 16, the presence or absence of inflammation of the gums or gingivae may be determined. In the same manner, a correlation between the degree of inflammation in the gums and gingivae and the intensity of the received light is calculated, and based on this correlation and the intensity of light received by the upper light receiving element 14 and the lower light receiving element 16, the degree of inflammation in the gums and gingiva may be determined. Further, the presence or absence and degree of inflammation in tissues around the gums and gingivae, that is, tissues in the deep part of the oral cavity 101 may be determined in the same manner. A detection result obtained in this manner is transmitted to the external device 60.

In the second embodiment also, as in the first embodiment, the biological information detection sensor 10 during measurement may be arranged so as not to move from a measurement position. This arrangement allows the biological information to be detected in the oral cavity 101 with high accuracy.

Note that the upper light emitting element 13, the upper light receiving element 14, the lower light emitting element 15, and the lower light receiving element 16 may be provided on an outer peripheral surface of the attachment portion 21 of the first embodiment. Furthermore, the light emitting element 11 and the light receiving element 12 of the first embodiment may be provided on an inner surface of the attachment portion 23 of the second embodiment.

### Third Embodiment

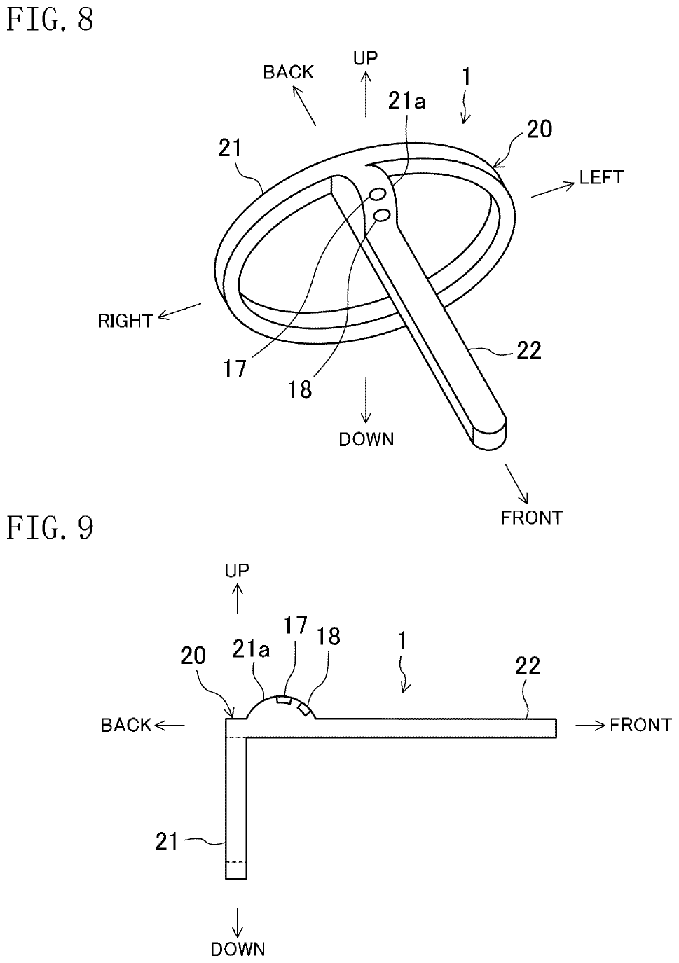

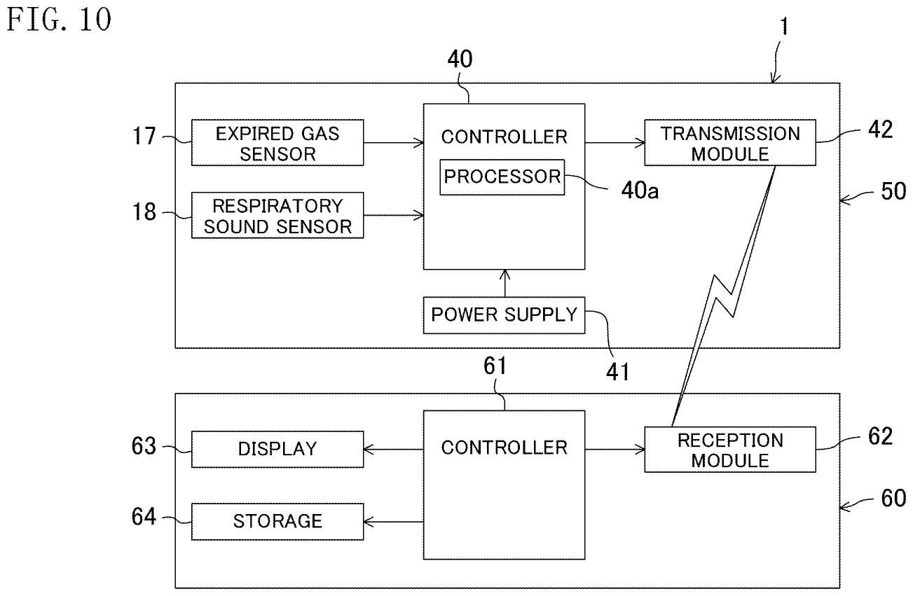

FIGS. 8 to 10 relate to a third embodiment of the present invention. The third embodiment is different from the first embodiment in that a biological information detector 1 is configured to detect expired gas and expiratory sound as biological information. Hereinafter, the same parts as those of the first embodiment are denoted by the same reference numerals, and the description thereof is omitted; different parts are described in detail.

As illustrated in FIGS. 8 and 9, an attachment portion 21 includes a bulge 21a projecting forward and bulging upward. An extending portion 22 is continuous from a front end of the bulge 21a. An expired gas sensor 17 and an expiratory sound sensor 18 that constitutes a biological information detection sensor 10 are provided on an upper surface of the bulge 21a. The expired gas sensor 17 is configured to detect a specific component contained in expiration of a subject 100, and is a typically known sensor. For example, it has been known that the expiration involving a certain disease contains a specific component. Thus, the disease may be identified by detecting the specific component contained in the expiration. The expired gas sensor 17 may also be configured to detect a plurality of components contained in the expiration. A detection result by the expired gas sensor 17 is output to a controller 40. A processor 40a may determine as follows based on the detection result of the expired gas sensor 17. When the specific component detected is, for example, a predetermined value or more, a disease is highly possibly involved. This determination result is displayed on a display 63.

The respiratory sound sensor 18 may be a microphone that detects the respiratory sound of the subject 100. The respiratory sound may also be a peculiar sound depending on the disease or physical condition. A detection result by the respiratory sound sensor 18 is output to the controller 40. The processor 40a may determine from the detection result of the respiratory sound sensor 18 that a disease is involved. In this case, a technique of obtaining a sound involving a disease and comparing the sound with a sound detected by the respiratory sound sensor 18 may be used. A determination result may be displayed on the display 63.

In the third embodiment also, as in the first embodiment, the biological information detection sensor 10 during measurement may be arranged so as not to move from a measurement position. This arrangement allows the biological information to be detected in the oral cavity 101 with high accuracy.

Note that only either of the expired gas sensor 17 or the expiratory sound sensor 18 may be provided. The expired gas sensor 17 and the expiratory sound sensor 18 may also be provided on the attachment portion 21 of the first and second embodiments.

### Fourth Embodiment

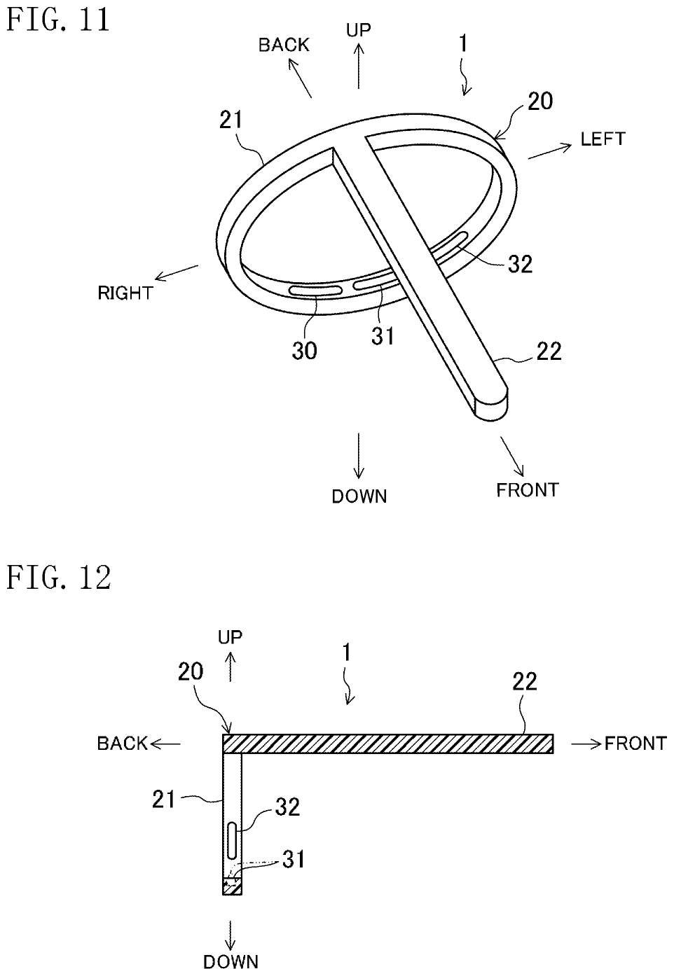

FIGS. 11 to 13 relate to a fourth embodiment of the present invention. The fourth embodiment is different from the first embodiment in that a biological information detector 1 is configured to detect a blood pressure as biological information. Hereinafter, the same parts as those of the first embodiment are denoted by the same reference numerals, and the description thereof is omitted; different parts are described in detail. In the fourth embodiment, the blood pressure may be detected by an oscillometric system used in a so-called electronic manometer, and the biological information detection sensor of the fourth embodiment is a blood pressure sensor.

As illustrated in FIGS. 11 and 12, in the fourth embodiment, a first inflation member 30, a second inflation member 31, and a third inflation member 32 are arranged in a circumferential direction on a lower side portion of an inner peripheral surface of an attachment portion 21. The first inflation member 30, the second inflation member 31, and the third inflation member 32 are pouch-shaped members formed from an elastic material such as rubber or elastomer, for example, and are configured to be inflated by injection of fluid such as air therein, and to be deflated by discharge of inner fluid. The attachment portion 21 of the fourth embodiment is formed from a member that does not expand or contract.

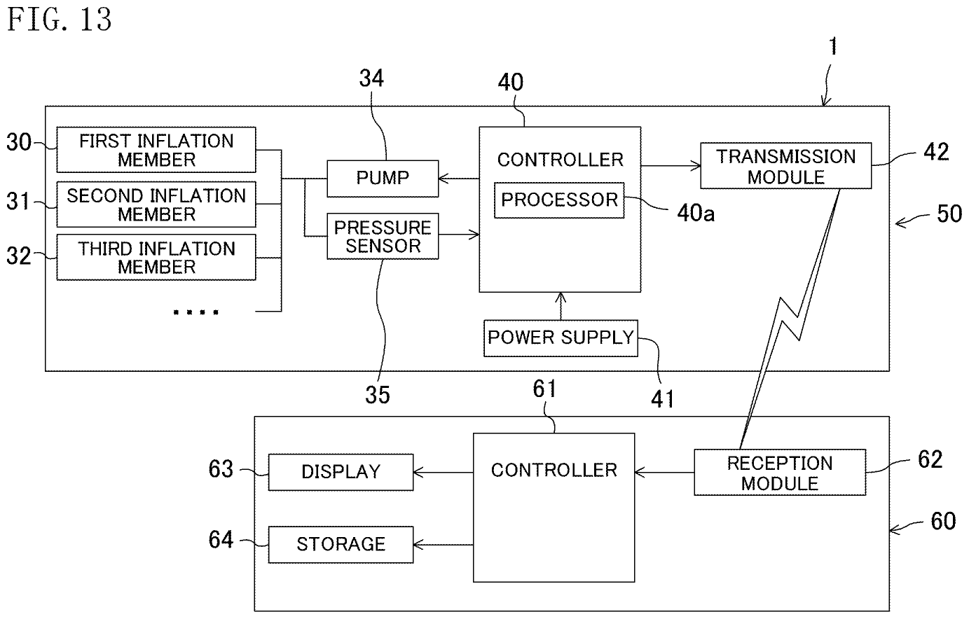

As illustrated in FIG. 13, in addition to the first inflation member 30, the second inflation member 31, and the third inflation member 32, a fourth inflation member and a fifth inflation member may also be provided. Furthermore, the number of inflation members may be one. For only one inflation member provided, the inflation member may have a continuous elongated shape in a right-to-left direction. Furthermore, the inflation member may also be provided on each of right and left sides.

A pump 34 capable of supplying and discharging air is connected to the first inflation member 30, the second inflation member 31, and the third inflation member 32. The pump 34 is provided outside an oral cavity 101, and the pump 34 is connected to the first inflation member 30, the second inflation member 31, and the third inflation member 32 by a pipe. The pipe may pass through the inside of an extending portion 22. The pump 34 is provided with a switching valve (not illustrated) that switches between a state in which an internal pressure chamber is open to the atmosphere and a state in which the switching valve is sealed, and this switching valve is also controlled by a controller 40. Note that the pump 34 may be a small pump that may be inserted into the oral cavity 101. In this case, the air (including the expiration) in the oral cavity 101 may be injected into the first inflation member 30, the second inflation member 31, and the third inflation member 32 by the pump 34.

By operating the pump 34, the air is injected into the first inflation member 30, the second inflation member 31, and the third inflation member 32 to inflate them. Since the attachment portion 21 is formed of an annular-shaped member that does not expand or contract, inflating directions of the first inflation member 30, the second inflation member 31, and the third inflation member 32 are regulated by the attachment portion 21, and these inflation members inflate only inward of the attachment portion 21.

A biological information detector 1 is provided with a pressure sensor 35 that detects internal pressures of the first inflation member 30, the second inflation member 31, and the third inflation member 32. The first inflation member 30, the second inflation member 31, the third inflation member 32, and the pressure sensor 35 constitute a biological information detection sensor. The pressure sensor 35 may be configured to detect the pressure in the pipe communicating with the first inflation member 30, the second inflation member 31, and the third inflation member 32, or configured to detect the inner pressure of any one of the first inflation member 30, the second inflation member 31, and the third inflation member 32. The pressure sensor 35 may be a typically known pressure sensor. A detection value of the pressure sensor 35 is output to the controller 40.

The controller 40 controls the pump 34. When, for example, a measurement start switch (not illustrated) connected to the controller 40 is operated with the attachment portion 21 attached to the tongue 102, the controller 40 operates the pump 34 to inflate the first inflation member 30, the second inflation member 31, and the third inflation member 32. When the first inflation member 30, the second inflation member 31, and the third inflation member 32 inflate, inflating force of the first inflation member 30, the second inflation member 31, and the third inflation member 32 is less likely to escape due to the attachment portion 21 in an annular shape, thereby allowing the first inflation member 30, the second inflation member 31, and the third inflation member 32 to reliably press the tongue 102. The first inflation member 30, the second inflation member 31, and the third inflation member 32 press a deep lingual artery. The deep lingual artery is the artery that extends toward a tip of the tongue 102 along a lower surface of the tongue 102. The degree of injection of air into the first inflation member 30, the second inflation member 31, and the third inflation member 32 may be determined based on the detection value of the pressure sensor 35. For example, control may be made to stop pressing when the blood flow at the pressed portion (deep lingual artery) stops.

This pressing stops the blood flow in the deep lingual artery. Thereafter, the controller 40 opens the pressure chamber of the pump 34, thereby gradually removing the air inside the first inflation member 30, the second inflation member 31, and the third inflation member 32. When the first inflation member 30, the second inflation member 31, and the third inflation member 32 are gradually deflated until the blood flows again into the deep lingual artery, a small heartbeat (pulse phenomenon) may be detected. This heartbeat may be detected based on the detection value of the pressure sensor 35. This pulsation becomes larger as tightening by the first inflation member 30, the second inflation member 31, and the third inflation member 32 becomes looser, reaches the largest amplitude, and then becomes smaller again. This change in pulsation may also be detected based on the detection value of the pressure sensor 35. The blood pressure may be calculated by analysis of amplitude waveform information of this pulsation with a predetermined algorithm. Specifically, since the blood pressure may be measured by the oscillometric system using the deep lingual artery, the blood pressure of a subject 100 having low blood pressure, which cannot be easily measured by a Korotkov's sound, may also be measured.

The oscillometric method may be used to measure systolic and diastolic blood pressures. After stopping the blood flow in a blood vessel, when the air inside the first inflation member 30, the second inflation member 31, and the third inflation member 32 is removed, the pulse occurs when the blood first flows, and vibration occurs. When the air inside the first inflation member 30, the second inflation member 31, and the third inflation member 32 is further removed, the blood vessel expands and an amount of blood flowing increases. Along with this, the vibration also increases, and after recording the maximum vibration, this gradually decreases to disappear. A time point when a vibration width rapidly increases may be regarded as the systolic blood pressure, and a time point when the vibration width rapidly decreases may be regarded as the diastolic blood pressure. Note that as the control method of the pump 34 and the analyzing method of the detection value of the pressure sensor 35 described above, the methods typically employed in the electronic manometer may be used.

In the fourth embodiment also, the first inflation member 30, the second inflation member 31, and the third inflation member 32 during measurement may be arranged so as not to move from the measurement position. This arrangement allows the blood pressure to be detected in the oral cavity 101 with high accuracy.

Note that the first inflation member 30, the second inflation member 31, the third inflation member 32, and the pressure sensor 35 of the fourth embodiment may be provided on the attachment portion 21 of the first to third embodiments. In this case, the pump 34 may be provided on the detection device 50 of the first to third embodiments.

### Fifth Embodiment

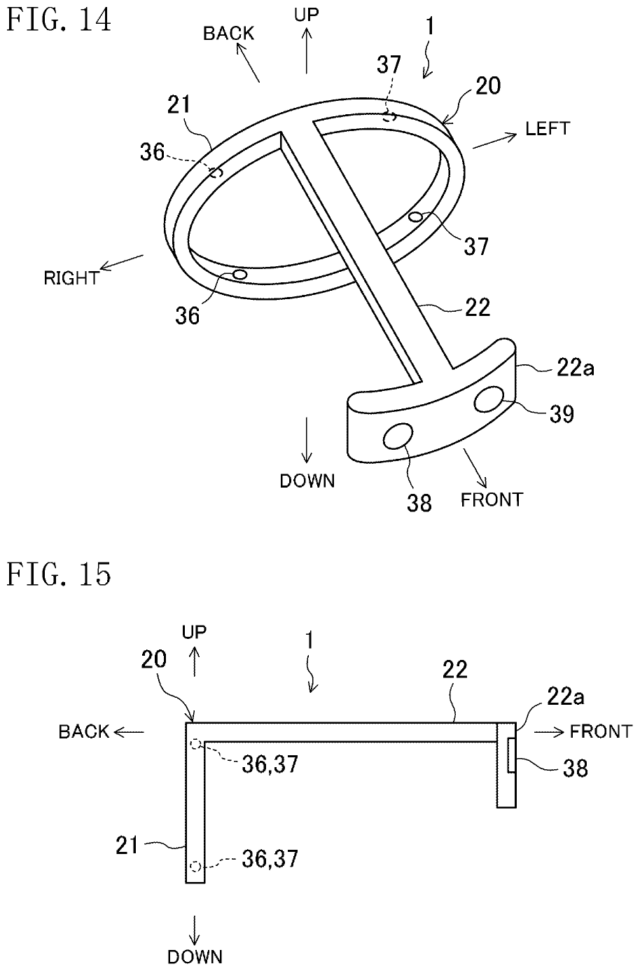

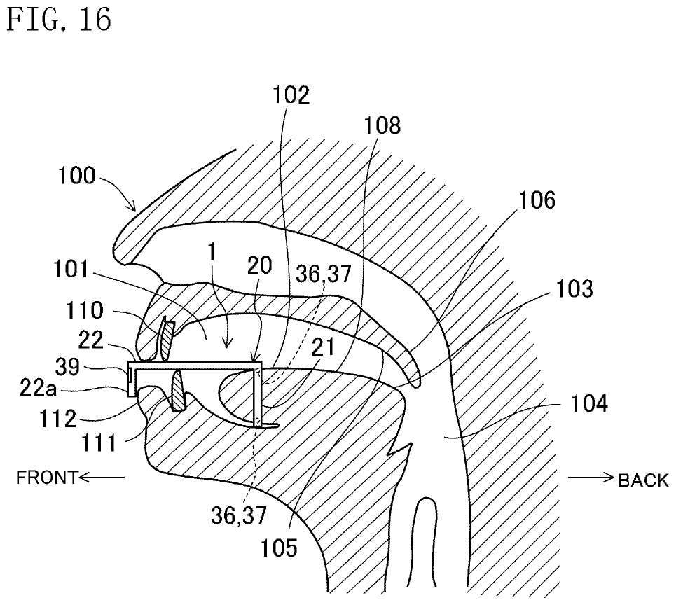

FIGS. 14 to 17 relate to a fifth embodiment of the present invention. The fifth embodiment is different from the first embodiment in that a biological information detector 1 is configured to detect a flow of electricity in the heart as biological information to obtain an electrocardiogram. Hereinafter, the same parts as those of the first embodiment are denoted by the same reference numerals, and the description thereof is omitted; different parts are described in detail. In the fifth embodiment, a biological information detection sensor is an electrocardiographic measurement sensor.

As illustrated in FIG. 14, the electrocardiographic measurement sensor includes a first intraoral electrode 36, a second intraoral electrode 37, a first extraoral electrode 38, and a second extraoral electrode 39. The first intraoral electrode 36 is provided on a right side of an inner peripheral surface of an attachment portion 21, and is arranged so as to come into contact with a right side of the tongue 102 when the attachment portion 21 is attached to the tongue 102. The second intraoral electrode 37 is provided on a left side of the inner peripheral surface of the attachment portion 21, and is arranged so as to come into contact with a left side of the tongue 102 when the attachment portion 21 is attached on the tongue 102.

An electrode mounting portion 22a is provided at a front end of an extending portion 22 so as to be located outside the oral cavity 101. The first extraoral electrode 38 is provided on a right side of the electrode mounting portion 22a, and the second extraoral electrode 39 is provided on a left side. The first extraoral electrode 38 is an electrode that comes into contact with a right hand of a subject 100. The second extraoral electrode 39 is an electrode that comes into contact with a left hand of the subject 100.

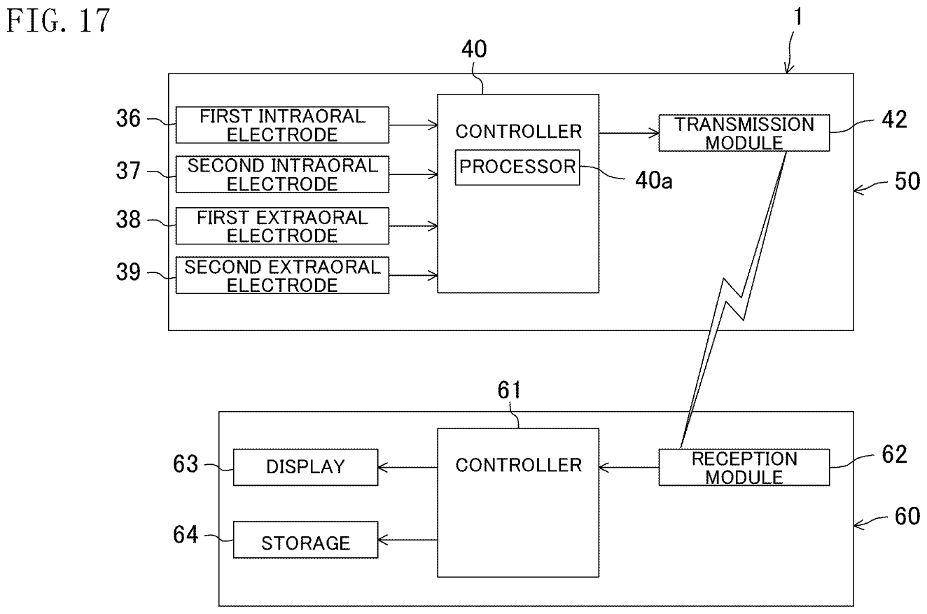

As illustrated in FIG. 16, when the attachment portion 21 is attached to the tongue 102, the first intraoral electrode 36 comes into contact with the right side of the tongue 102 and the second intraoral electrode 37 comes into contact with the left side of the tongue 102. Furthermore, the first extraoral electrode 38 and the second extraoral electrode 39 are arranged outside the oral cavity 101, and the subject 100 may come into contact with the first extraoral electrode 38 and the second extraoral electrode 39 with the right hand and the left hand, respectively. As illustrated in FIG. 17, the first intraoral electrode 36, the second intraoral electrode 37, the first extraoral electrode 38, and the second extraoral electrode 39 are connected to a controller 40. The controller 40 calculates a change in voltage detected by the first intraoral electrode 36, the second intraoral electrode 37, the first extraoral electrode 38, and the second extraoral electrode 39 to generate the electrocardiogram. Specifically, the biological information detector 1 is configured to obtain the electrocardiogram with few electrodes by utilizing the Einthoven's triangle theorem. As described above, by bringing the electrodes into contact with three of the tongue 102, the right hand, and the left hand, three bipolar-lead electrocardiograms may be obtained. The electrode brought into contact with one site serves as a positive electrode and a negative electrode. Therefore, when there electrodes at the respective three sites, an imaginary electrode (indifferent electrode) is formed at the center thereof. It is possible to obtain the electrocardiograms by a unipolar-lead method between this indifferent electrode as a starting point and the above-described electrodes at the three sites.

In the fifth embodiment, the first intraoral electrode 36 and the second intraoral electrode 37 during measurement may be arranged so as not to move from measurement positions. This arrangement allows biological information to be detected in the oral cavity 101 with high accuracy.

The embodiments described above are mere examples in every respect, and shall not be interpreted in a limited manner. Variations and modifications of equivalents of the claims are all intended to fall within the scope of the present disclosure. For example, the biological information detector 1 of the first to fifth embodiments may be provided with a temperature sensor that detects body temperature. Furthermore, the biological information detector 1 of the first to fifth embodiments may be provided with a detector that detects saliva components. The detector is a sensor configured to detect the saliva components (e.g., proteins, carbohydrates, fats, glucose, various cancer markers). By analyzing the components in saliva and measuring a level of each biomarker, various symptoms may be detected early. Moreover, the saliva contains glucose by an amount much smaller than that of blood, and it is possible to estimate a blood glucose level by providing a sensor capable of measuring an amount of glucose contained in the saliva. Specifically, diabetes may be diagnosed by collecting the saliva instead of blood. As the biomarker and the method of measuring glucose, the methods described in various academic documents and the like may be used. In this case, examples of the detector may include a light emitter and the one that generates magnetic force.

### INDUSTRIAL APPLICABILITY

As described above, the present invention may be used, for example, to obtain vital data such as an arterial blood oxygen saturation, a pulse wave, a blood pressure, an expired gas, a respiratory sound, and a degree of inflammation in gums and gingivae.

### DESCRIPTION OF REFERENCE CHARACTERS

1 Biological Information Detector

10 Biological Information Detection Sensor

11 Light Emitting Element (Light Emitter)

12 Light Receiving Element (Light Receiver)

17 Expired Gas Sensor

18 Expiratory Sound Sensor

20 Sensor Holder

21 Attachment Portion

22 Extending Portion

30 Inflation Member

34 Pump

35 Pressure Sensor

36 Intraoral Electrode

38 Extraoral Electrode

50 Detection Device

1. A biological information detector comprising:
a biological information detection sensor configured to be inserted into an oral cavity and detect biological information in the oral cavity, and
a sensor holder holding the biological information detection sensor, wherein
the sensor holder includes an attachment portion continuous to surround a front surface, lateral surfaces, and a back surface of a tongue of a subject, and is used to attach the tongue.

2. The biological information detector of claim 1, wherein
the sensor holder further includes an extending portion continuous from the attachment portion toward an area between upper and lower teeth of the subject.

3. The biological information detector of claim 1, wherein
the attachment portion has an annular shape continuous to surround the front surface, a left lateral surface, the back surface, and a right lateral surface of the tongue of the subject.

4. The biological information detector of claim 3, wherein
an inflation member that presses the tongue is arranged in an inner peripheral surface of the attachment portion, and
the biological information detection sensor is a blood pressure sensor including the inflation member.

5. The biological information detector of claim 1, wherein
the biological information detection sensor includes a light emitter that irradiates the tongue with light, and a light receiver that receives the light applied to the tongue from the light emitter, the light emitter and the light receiver being arranged to come into contact with the back surface of the tongue.

6. The biological information detector of claim 1, wherein
the biological information detection sensor includes a light emitter that irradiates a gum with light, and a light receiver that receives the light applied to the gum from the light emitter, the light emitter and the light receiver being arranged so as to face the gum.

7. The biological information detector of claim 1, wherein
the biological information detection sensor is an electrocardiographic measurement sensor including an intraoral electrode arranged to come into contact with the tongue and an extraoral electrode arranged outside the oral cavity to come into contact with a hand of the subject.

8. The biological information detector of claim 1, wherein
the biological information detection sensor is an expired gas sensor or a respiratory sound sensor.
